(19) United States
(12) Patent Application Publication
(10) Pub. No.: US 2007/0118218 A1
(43) Pub. Date: May 24, 2007

(54) Facet joint implant and procedure

(76) Inventor: David M. Hooper, Austin, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/287,077

(22) Filed: Nov. 22, 2005

(51) Int. Cl. A61F 2/28

(52) U.S. Cl. 623/14.12

(57) ABSTRACT

A method for repairing a facet joint of a human vertebra having a joint capsule surrounding the facet joint. In the method, a synthetic elastic material is introduced into the facet joint. The synthetic elastic material can be a solid, swellable polymer that expands when hydrated upon being placed in the facet joint. The invention includes the implant and the method of making the implant.

### BACKGROUND OF THE INVENTION

This invention relates to a facet joint implant and a method for distracting a facet joint and maintaining separation of the facet joint.

The facet joints, knees, and elbows are sometimes referred to as synovial joints. A synovial joint allows movement between two bones. In a synovial joint, the ends of the bones are covered with a material called articular cartilage. This material is a slick spongy material that allows the bones to glide against one another without much friction.

Surrounding the facet joint is a watertight sack made of soft tissue and ligaments. This sack creates what is called the “joint capsule”. The ligaments are soft tissue structures that hold the two sides of the facet joint together. The ligaments around the facet joint combine with the synovium to form the joint capsule that is filled with fluid (synovial fluid). This fluid lubricates the joint to decrease the friction.

The facet joint can often become painful during the degenerative process in the spine. Loss of disc height can reduce the separation of opposing facet joints and alters the biomechanics of those joints. The cartilage of the joint may become compromised or destroyed resulting in bone on bone contact in the joint. This may cause significant pain.

Currently, this type of pain is treated by anesthetic injections or surgical destruction of the nerves.

### SUMMARY OF THE INVENTION

This invention provides a solution to one or more of the deficiencies and disadvantages described above.

In one broad respect, this invention is a method for repairing a facet joint of a human vertebra having a joint capsule surrounding the facet joint, comprising: introducing a synthetic elastic material into the facet joint. The synthetic elastic material can be introduced through the joint capsule into the facet joint or, alternatively, introduced through the bone into the facet joint to thereby maintain separation of the facet joint. In one representative embodiment, the synthetic elastic material can be a hydrogel. The synthetic elastic material can be introduced as a fully polymerized implant or, alternatively, as a polymerizable composition that polymerizes to form the hydrogel within the facet joint. The synthetic elastic material, in one embodiment, can be in a dehydrated or partially dehydrated form prior to introduction into the facet joint, and which swells upon hydration in the facet joint. In one embodiment, the method includes distracting the facet joint prior to introduction of the synthetic elastic material. In one embodiment, the synthetic elastic material is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials.

In another broad respect, this invention is a method for repairing a facet joint of a human vertebra having a joint capsule surrounding the facet joint, comprising: introducing a solid swellable synthetic elastic material into the facet joint. In one embodiment, the synthetic elastic material is introduced through the joint capsule into the facet joint. In one embodiment, the synthetic elastic material is introduced through bone into the facet joint. In one embodiment, the method includes distracting the facet joint prior to introduction of the synthetic elastic material. In one embodiment, the elastic material swells after being introduced into the facet joint. In one embodiment, the synthetic elastic material is introduced in the form of a folded or rolled solid swellable polymerized implant. In one embodiment, the synthetic elastic material is an elastomer that is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials.

In another broad respect, this invention is a method for repairing a facet joint of a human vertebra having a joint capsule surrounding the facet joint, comprising: introducing a polymerizable composition into the facet joint, wherein the polymerizable composition forms a synthetic elastic material in the facet joint. In one embodiment, the polymerizable composition is introduced through the joint capsule into the facet joint. In one embodiment, the polymerizable composition is introduced through bone into the facet joint. In one embodiment, the method includes distracting the facet joint prior to introduction of the polymerizable composition. In one embodiment, the synthetic elastic material is initially formed as a swellable polymerized composition which swells in the facet joint. In one embodiment, the synthetic elastic material is an elastomer that is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials.

In another broad respect, this invention is an implant comprising a solid synthetic elastic material and adapted for use as a facet joint implant. The implant is thus of a size and dimensions during use that allow it to be used as a facet joint implant. The synthetic elastic material can be in the form of a swellable polymerized composition. The synthetic elastic material can be formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials. In one embodiment, the synthetic elastic material is in the form of a hydrogel.

In another broad respect, this invention is a method for manufacturing a facet implant, comprising forming a synthetic elastic material into an implant adapted for use as a facet implant in a human spine.

This invention provides a number of advantages, including but not limited to the following. The synthetic elastic material employed in the practice of this invention provides a surface to enhance lubrication within the facet joint, which can reduce pain associated with degenerated facet joints. The elastic material is relatively inexpensive. The method introduces the elastic material into the facet joint in a relatively non-invasive procedure. The elastic material is advantageously benign, biocompatible, elastic, and pliable, and can be formed from synthetic polymers previously used in the human body. Thus, at least some of the polymers that can be used in the practice of this invention are advantageously commercially available. When a solid elastic material is introduced into the facet joint, the elastic material can be introduced as an at least partially dehydrated solid in a shape that conforms to the cavity within the facet joint. In this regard, the at least partially dehydrated solid becomes re-hydrated after being introduced into the facet joint. The elastic material can thus swell to a larger size than the incision or hole that the elastic material is introduced through, thereby preventing the swelled elastic material from undesirably becoming expelled from the facet joint. Beneficially, the elastic material can be readily removed if, for example, it is later desired to remove the facet joint if a spinal fusion procedure is performed.

### BRIEF DESCRIPTION OF THE DRAWINGS

FIG. 1 illustrates a cross-sectional view of a facet joint, with the polymerizable composition being injected into the facet joint through the joint capsule.

FIGS. 2A, 2B, and 2C illustrate representative views of a synthetic elastic material being introduced into a facet joint through an incision in the joint capsule.

FIG. 3 illustrates a representative view of a synthetic elastic material being introduced into a facet joint through a hole in the bone.

FIGS. 4A-4K illustrate representative shapes of solid synthetic elastic material that can be introduced into a facet joint according to this invention.

FIG. 5 illustrates a synthetic elastic material within a facet joint depicted in as introduced and swelled forms.

### DETAILED DESCRIPTION OF THE INVENTION

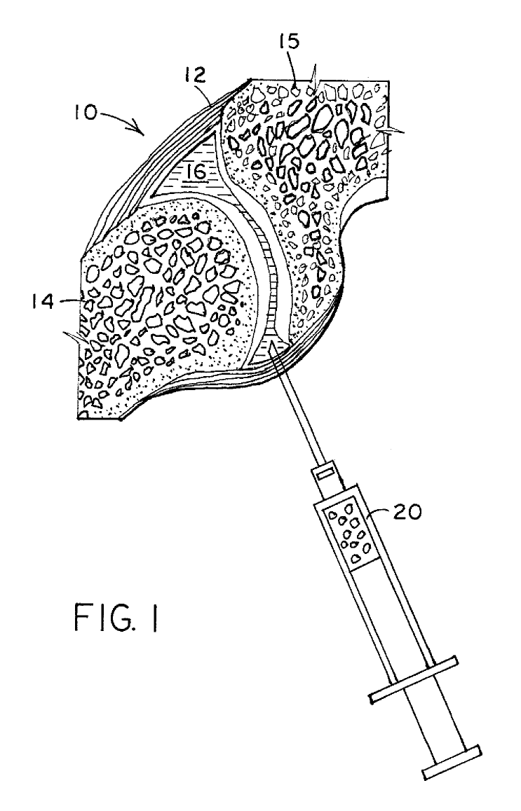

FIG. 1 illustrates a cross-section of a facet joint 10. The facet joint 10 includes the joint capsule 12 that attaches to the bone 14, 15 of an upper and lower vertebra. The joint capsule 12 and bones 14, 15 together define an inner cavity 16 that normally holds synovial fluid. Thus, the joint capsule 12 surrounds the inner cavity on the perimeter, and the bones 14, 15 define the upper and lower ends of the inner cavity 16. The synovial fluid provides lubrication for the facet joint. If the facet joint degenerates, there can be a lessoning of synovial fluid, reduction in space between the bones 14, 15 such that painful bone-on-bone contact occurs. The present invention provides a synthetic elastic material of appropriate shape and size to be placed in the cavity 16 so that bone-on-bone contact is reduced or eliminated, thereby reducing or eliminating pain for a patient. The implant may also provide lubrication for the facet joint.

After it is determined that a facet joint is in need of the procedure discussed herein, it should be determined whether the facet joint should be distracted prior to introduction of the synthetic hydrogel into the facet joint 10. If needed, such as the size of the cavity is insufficient to allow introduction of the hydrogel, the distraction of the facet joint can be accomplished through techniques well known to one of skill in the art. In general, the distraction can be accomplished, for example, by wedging the facet joint apart, such as by using a ramped needle, screws, a wedge, an osteotome, or some specific delivery device.

Next, a synthetic elastomeric material is introduced into the cavity of the facet joint. The term “synthetic elastomeric material” refers to man-made materials such polymers, as opposed to naturally occurring materials such as collagen, naturally occurring proteins, cartilage and so on. In one embodiment, the synthetic elastomeric material is a hydrogel. As is known, hydrogels attract water. In general, the hydrogels used in the practice of this invention contain at least 25 percent by weight of water when fully hydrated and which contain this quantity of water in the facet joint. In one embodiment, the hydrogels contain at least 50 percent by weight of water and in certain embodiments contain at least 90 percent by weight of water. The hydrogels in general are inert, solid, elastic, pliable and biocompatible. The synthetic elastomeric material, such as a hydrogel, introduced into the facet joint provides relief from the facet joints rubbing each other, and may provide lubrication between the joints. The synthetic elastomeric material, including a hydrogel, can be fully hydrated when introduced into the facet joint, or can be, for example, introduced as a swellable material (e.g., a dehydrated sheet) that attracts water and swells/rehydrates once introduced into the joint.

The synthetic elastic material can be in the form of a polymerizable composition to be introduced into the facet joint or a fully polymerized composition. The final polymers can be cross-linked or not cross-linked. It should be appreciated that the polymerizable composition and the fully polymerized elastic material can be made from the same monomers and/or polymer precursors.

A polymerizable composition can be introduced into the cavity, which fully polymerizes within the cavity. The polymerizable material can be partially polymerized prior to introduction into the cavity. Depending on the type of polymerizable composition, a polymerization catalyst or initiator may be needed. In one embodiment, the polymerizable composition polymerizes in the facet joint to form an expandable elastic material, which swells up until its equilibrium water content is reached, i.e., a dehydrated elastic material is introduced into the cavity and swells as it rehydrates. The elastic material may be a hydrogel. However, the elastic material formed from the polymerizable composition in the joint need not necessarily be swellable to be useful in the practice of this invention.

As shown in FIG. 1, the polymerizable composition can be introduced into the facet joint 10 such as by injection using a suitable syringe fitted with a hypodermic needle 20 or cannula. In some cases it may be desirable to use a dual barrel syringe, where one syringe holds the monomers and/or polymer precursors components and the second syringe holds a catalyst or initiator composition, which mix in the connector of the syringe or in the needle or in the body whereupon polymerization occurs. The hole in the joint capsule caused by the needle or in the bone may heal naturally or may be sutured, patched, or filled with a suitable material to seal the hole. For example, if bone is drilled to create a conduit for introduction of the synthetic elastic material (whether a polymerizable composition or fully polymerized prior to introduction into the facet joint), the bone can be filled with bone material, adhesive, or other filler, then capped or plugged. Alternatively, a metal screw or a screw formed of a material that forms bone over time can be used to close the drilled conduit. In one embodiment, the polymerizable composition can be injected into a balloon that has been previously placed in the facet joint. The balloon, or some other containment system, serves to contain the polymerizable composition as it is injected.

Alternatively, a fully polymerized synthetic elastic material can be introduced into the facet joint. In one embodiment, the polymer is an expandable synthetic elastic material (e.g., a hydrogel), which swells until its equilibrium water content is reached. For instance, a dehydrated synthetic elastic material is introduced into the cavity and swells as it rehydrates. If the synthetic elastic material used is a fully polymerized polymer to be introduced into the facet joint, it can be in the form of particles, or be in other forms such as in the form of a sheet or elongate rods (e.g., that resemble toothpicks). The sheet or elongate rods can be inserted through a minimally invasive hole either through the joint capsule or through the bone. If the sheet or elongate rods absorb water, the sheet or elongate rods expand upon absorbing water within the facet joint, thus creating a larger sheet or rod that will not exit the cavity through the hole in either the bone or the joint capsule. The implant, whether a sheet or otherwise, is of a size and shape adapted to be inserted into the cavity of the facet joint. In one embodiment, the sheet is inserted in a dehydrated, reduced size such as in a folded, coiled, wrapped, or rolled shape, which upon hydration opens into a sheet within the facet joint.

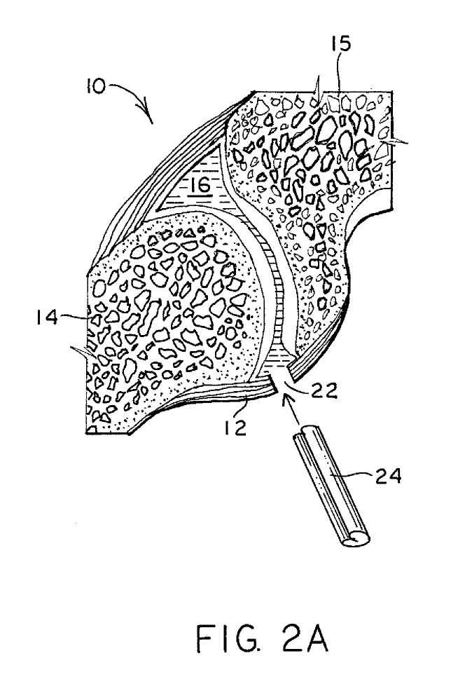

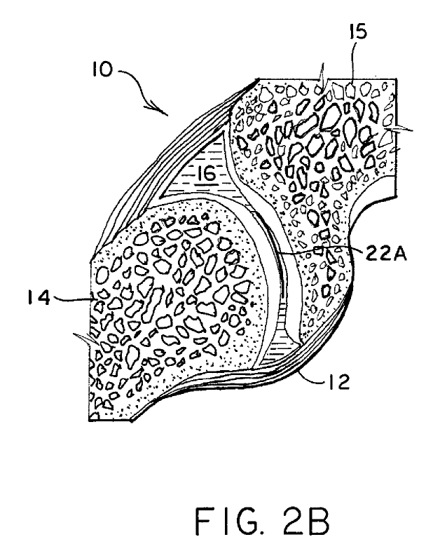

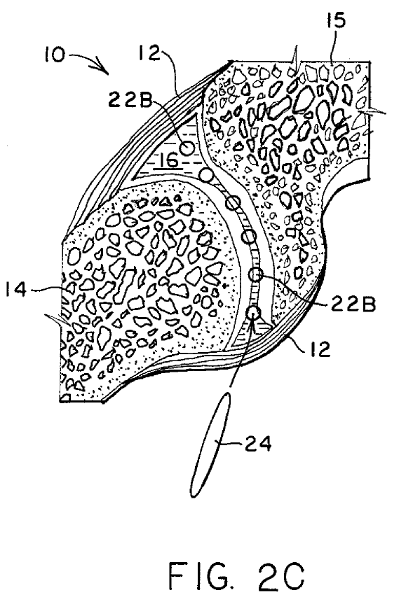

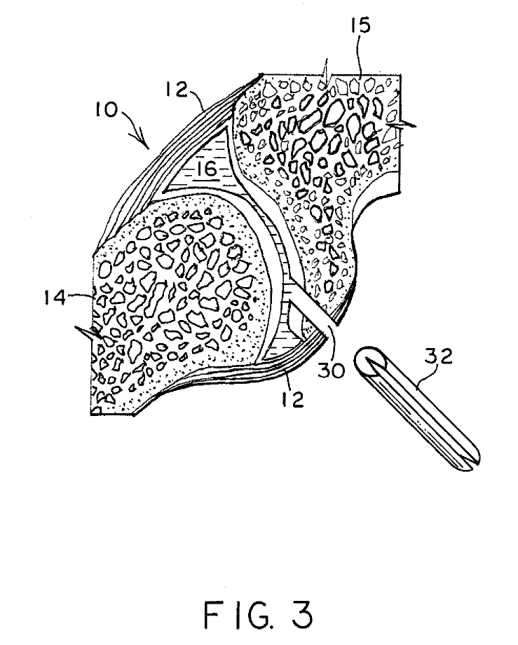

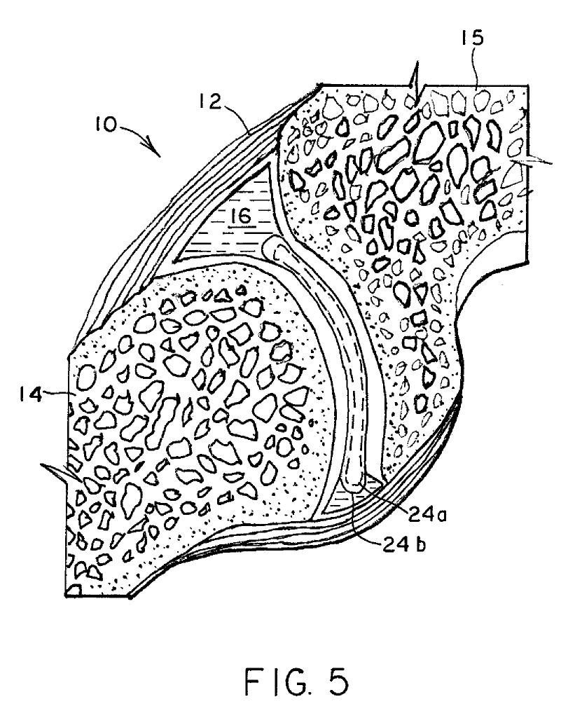

In the case of a fully polymerized, solid synthetic elastic material implant, an incision 22 can be made in the joint capsule 12 such as depicted in FIG. 2. The incision, such as in the shape of a round hole, is of sufficient size to permit the insertion of the implant 24 into the cavity 16 within the facet joint 10. FIG. 5 illustrates a synthetic elastic material within a facet joint depicted in as introduced form 24a (in dotted lines) and swelled form 24b. It should be appreciated that the sizes depicted in the figures may not be to scale. In another embodiment, a slice 22A is made in the joint capsule 12 to provide an entrance incision for the synthetic elastic material, as depicted in FIG. 2B. Alternatively, a rectangular hole can be made in the bone with an implant in the form of, for example, a sheet slid into the facet joint. In another embodiment, the implant 24 is in the shape of an elongate rod (e.g., “toothpick shaped”) as depicted in FIG. 2C that is inserted through holes 22B cut at multiple points around the capsule 12. The elongate implant 24 in FIG. 2C may swell within the joint to a larger size. It should be appreciated that one or more rods can be inserted in the facet joint through one or more incisions. Alternatively, as depicted in FIG. 3, a hole 30 can be drilled through bone 15 to permit the insertion of the implant 32 in the cavity 16. In either case the incision or hole can be optionally sealed after the implant has been introduced into the facet joint. Advantageously, if a dehydrated implant is introduced into the facet joint, and then the implant is hydrated to facilitate swelling, if sized appropriately the implant will not exit through the incision or hole in the bone, whether or not the incision or hole is subsequently sealed by the physician. If small synthetic elastic materials in the form of particles are used, under some circumstances it may be possible to introduce these particles into the facet joint such as by injection using a suitable syringe fitted with a hypodermic needle. The hole in the joint capsule caused by the needle or in the bone may heal naturally or may be sutured, patched, or filled with a suitable material. For example, if bone is drilled to create a conduit for introduction of the synthetic elastic materials (whether a polymerizable composition or fully polymerized prior to introduction into the facet joint), the bone can be filled with bone material, adhesive, or other filler, then capped or plugged. Alternatively, a metal screw or a screw formed of a material that forms bone over time can be used to close the drilled conduit.

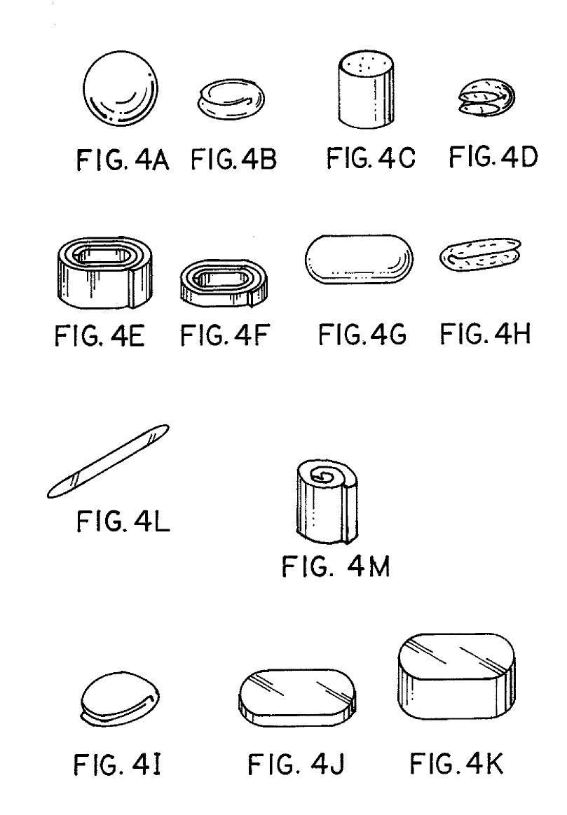

Representative shapes of solid synthetic elastic materials are shown in FIGS. 4A-4M. A representative elastic material in the shape of a sphere in hydrated form is depicted in FIG. 4A, with the sphere in a dehydrated, folded form depicted in FIG. 4B. A cylindrical shape is depicted in FIG. 4C in its hydrated form, and in its dehydrated, folded form in FIG. 4D. FIG. 4E shows a hydrated helix with FIG. 4F showing the helix in dehydrated form. An implant of a hydrated, ovoid shape is depicted in FIG. 4G, with a folded, dehydrated ovoid depicted in FIG. 4H. FIG. 4I depicts a folded, dehydrated oblong sheet with FIG. 4J depicting a dehydrated oblong sheet that is not folded. FIG. 4K depicts a rehydrated oblong sheet, formed by hydration of the shape in either FIG. 4I or FIG. 4J. FIG. 4L depicts an elongate rod, which can be inserted into the joint. In one embodiment the elongate rod hydrates to expand within the joint. FIG. 4M shows a sheet in the form of a roll, which unfurls within the joint to form a sheet. In each of FIGS. 4A-4M, the height, width, and depth separately in each occurrence of the shapes can vary widely depending on the size of the joint for a given person at the given part of the spine. Typically, the area to be treated is believed to be approximately 120 square millimeters. The shapes can also include rectangles, ovals, and circles. The thickness of the implants can vary, such as being less than 2 millimeters when dehydrated, and about 2 to about 3 millimeters in the absence of a compressive load. In the case of the elongate rod of FIG. 4L, the rods are typically about 3 to about 15 millimeters in length and a diameter of less than 1 millimeter. The rods can be inserted through a needle and then rehydrated in the joint. The rods can have tapered or blunt ends. In one embodiment, a single implant is introduced into the facet joint. In another embodiment, two or more implants are inserted into the facet joint, such as for example in the case of multiple, small spheres, rods, or other particles being inserted or injected into the cavity. The shapes depicted in FIGS. 4A-4K are intended to be representative. Other shapes and sizes can be used.

The polymers that can be used in the practice of this invention to make the polymerizable compositions and polymerized elastic materials (including hydrogels) include but are not limited to polyacrylonitrile, polyvinyl alcohol, polyvinylpyrrolidone, polyacrylic acid, polymethacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, (pliable) epoxy polymers, and combinations thereof, as well as the monomers used to make such polymers. The polymers and copolymers of this invention can be made of monomers such as but not limited to that can be employed to make the polymers used in this invention include but are not limited to hydroxyalkyl acrylates such as 2-hydroxy ethyl methacrylate, acrylic acid, acrylonitrile, urea, ethylene oxide and propylene oxide, acrylamide, tetrafluoroethylene, dimethylsiloxane, monomers used to form polyurethane such as polyols and diisocyanates such as diphenylmethane diisocyanate (MDI), monomers used to form pliable epoxy resins, vinyl alcohol, methacrylates including alkyl methacrylates such as methyl methacrylate, N-vinyl monomers such as N-vinyl-2-pyrrolidone, ethylenically unsaturated acids such as methacrylic acid, ethylenically unsaturated bases such as 2-(diethylamino) ethyl methacrylate. The polymers can be made using well known techniques, and may be commercially available. Likewise, polymers can be readily formed into sheets and so on, as described herein, using well known techniques.

In general, if monomers and/or polymer precursors are introduced into the cavity, the monomers and/or polymer precursors react in the body to form the final polymeric composition. As used herein, “polymer precursor” (which can also be referred to as a “prepolymer”) refers to materials that are formed by the partial polymerization of monomers, such as to form chains by reaction of, for example, two to four monomer groups.

In some cases, depending on the type of monomers or polymer precursors employed, polymerization initiators or catalysts are required to cause polymerization. Such compounds can be, for example, free radical initiators. In other cases, heat or light (e.g., UV light) can serve to initiate polymerization.

Representative examples of suitable polymeric materials are described in U.S. Pat. No. 5,976,186, U.S. Pat. No. 6,264,695, U.S. Pat. No. 6,280,475, U.S. Pat. No. 6,443,988, and U.S. Pat. No. 6,595,998, each of which is incorporated herein by reference in their entirety.

The synthetic elastic materials can contain a variety of other additives, such as pharmaceutically active compounds, analgesics, antibiotics, nutrients, building blocks for tissue generation, and so on. Likewise, a lubricating composition may be introduced concurrent with the synthetic elastic materials, such as additional synovial fluid, hyaluronic acid, and so on. Also, the implants can include radiographic markers such as strips of tantalum wire.

Further modifications and alternative embodiments of this invention will be apparent to those skilled in the art in view of this description. Accordingly, this description is to be construed as illustrative only and is for the purpose of teaching those skilled in the art the manner of carrying out the invention. It is to be understood that the forms of the invention herein shown and described are to be taken as illustrative embodiments. Equivalent elements or materials may be substituted for those illustrated and described herein, and certain features of the invention may be utilized independently of the use of other features, all as would be apparent to one skilled in the art after having the benefit of this description of the invention.

What is claimed is:

1. A method for repairing a facet joint of a human vertebra having a joint capsule surrounding the facet joint, comprising: introducing a synthetic elastic material into the facet joint.

2. The method of claim 1, wherein the synthetic elastic material is introduced through the joint capsule into the facet joint.

3. The method of claim 1, wherein the synthetic elastic material is introduced through bone into the facet joint.

4. The method of claim 1, further comprising distracting the facet joint prior to introduction of the synthetic elastic material.

5. The method of claim 1, wherein the synthetic elastic material is introduced as a polymerizable composition that polymerizes in the facet joint.

6. The method of claim 1, wherein the synthetic elastic material is introduced as a polymerized implant.

7. The method of claim 1, wherein the synthetic elastic material is introduced as a swellable polymerized implant.

8. The method of claim 1, wherein the synthetic elastic material is introduced in the form of a folded swellable polymerized implant.

9. The method of claim 1, wherein the synthetic elastic material is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials.

10. The method of claim 1, wherein the synthetic elastic material is in the form of a hydrogel.

11. The method of claim 1, wherein the synthetic elastic material contains a pharmaceutically active compound, an analgesic, an antibiotic, a nutrient, a building block for tissue generation, and a combination thereof.

12. A method for repairing a facet joint of a human vertebra having a joint capsule surrounding the facet joint, comprising: introducing a solid swellable synthetic elastic material into the facet joint.

13. The method of claim 12, wherein the synthetic elastic material is introduced through the joint capsule into the facet joint.

14. The method of claim 12, wherein the synthetic elastic material is introduced through bone into the facet joint.

15. The method of claim 12, further comprising distracting the facet joint prior to introduction of the synthetic hydrogel.

16. The method of claim 12, wherein the hydrogel swells after being introduced into the facet joint.

17. The method of claim 12, wherein the synthetic hydrogel is introduced in the form of a folded solid swellable polymerized implant.

18. The method of claim 12, wherein the synthetic hydrogel is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, and combinations thereof.

19. The method of claim 12, wherein the synthetic elastic material contains a pharmaceutically active compound, an analgesic, an antibiotic, a nutrient, a building block for tissue generation, and combinations thereof.

20. The method of claim 12, wherein the synthetic elastic material is in the form of a hydrogel.

21. A method for repairing a facet joint of a human vertebra having a joint capsule surrounding the facet joint, comprising: introducing a polymerizable composition into the facet joint, wherein the polymerizable composition forms a synthetic elastic material in the facet joint.

22. The method of claim 21, wherein the polymerizable composition is introduced through the joint capsule into the facet joint.

23. The method of claim 21, wherein the polymerizable composition is introduced through bone into the facet joint.

24. The method of claim 21, further comprising distracting the facet joint prior to introduction of the polymerizable composition.

25. The method of claim 21, wherein the synthetic elastic material is initially formed as a swellable polymerized composition which swells in the facet joint.

26. The method of claim 21, wherein the synthetic hydrogel is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, and combinations thereof.

27. The method of claim 21, wherein the synthetic elastic material is in the form of a hydrogel.

28. The method of claim 21, wherein the synthetic elastic material contains a pharmaceutically active compound, an analgesic, an antibiotic, a nutrient, a building block for tissue generation, or a combination thereof.

29. An implant comprising a solid synthetic elastic material and adapted for use as a facet joint implant.

30. The implant of claim 29, wherein the synthetic elastic material is in the form of a swellable polymerized composition.

31. The implant of claim 29, wherein the synthetic elastic material is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, and combinations thereof.

32. The implant of claim 29, wherein the synthetic elastic material is in the form of a hydrogel.

33. The implant of claim 29, wherein the synthetic elastic material contains a pharmaceutically active compound, an analgesic, an antibiotic, a nutrient, a building block for tissue generation, or a combination thereof.

34. A method for manufacturing a facet implant, comprising: forming a synthetic elastic material into an implant adapted for use as a facet implant in a spine.

35. The method of claim 34, wherein a polymerized implant.

36. The method of claim 34, wherein the synthetic elastic material is a swellable polymerized implant.

37. The method of claim 34, wherein the synthetic elastic material is a folded swellable polymerized implant.

38. The method of claim 34, wherein the synthetic elastic material is formed from polyacrylonitrile, polyvinyl alcohol, polyvinyl pyrrolidone, polyacrylic acid, poly methacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, epoxy polymers, or a combination of one or more of these materials.

39. The method of claim 34, wherein the synthetic elastic material is in the form of a hydrogel.

40. The method of claim 34, wherein the synthetic elastic material contains a pharmaceutically active compound, an analgesic, an antibiotic, a nutrient, a building block for tissue generation, and a combination thereof.
